(19) United States
(12) Patent Application Publication
(10) Pub. No.: US 2014/0238577 A1
(43) Pub. Date: Aug. 28, 2014

(54) Adjustable Pant-Like Disposable Undergarment With Fully Severed Front Panel And Method Of Manufacture With Product Defect Detection System

(71) Applicant: KIMBERLY-CLARK WORLDWIDE, INC., Neenah, WI (US)

(72) Inventors: Jason Andrew Verboomen, Appleton, WI (US); Bradley William Schoon, Oshkosh, WI (US); Brian Keith Rhodes, Larsen, WI (US); Robert Lee Popp, Greenville, WI (US); Patrick August Lorsung, Amherst, WI (US); Paul George Milbrodt, Neenah, WI (US); Marlene Ruth Dins, Appleton, WI (US); Joseph A. Mlinar, Appleton, WI (US)

(73) Assignee: KIMBERLY-CLARK WORLDWIDE, INC., Neenah, WI (US)

(21) Appl. No.: 13/780,485

(22) Filed: Feb. 28, 2013

(51) Int. Cl. A61F 13/15

(52) U.S. Cl. CPC A61F 13/15772; A61F 13/15756
USPC 156/64; 156/378

(57) ABSTRACT

A method that includes conveying a moving web for use as a body panel on a web stability control module is disclosed. A fastener assembly is supplied that may be used to attach and connect portions of the moving web. Prior to attaching the fastener assembly to the moving web, a detection system is utilized to determine if there is a product defect on the fastener assembly. If no product defect is detected, the moving web is cut at a cutting station to completely separate a leading portion of the moving web and a trailing portion of the moving web. Then, the fastener assembly is attached to the moving web at a fastener assembly attachment station such that the leading portion and trailing portion remains bridged together. If a product defect is detected, then the moving web is not cut, and the discrete disposable absorbent undergarment is discarded.

### BACKGROUND

Pant-like disposable undergarments for absorbing human discharges can appear similar in size and shape to regular cloth underwear which is designed to be laundered and reused. A disposable absorbent undergarment is intended to be worn by persons, including infants, toddlers, or adults, and is designed for a single or temporary use and is meant to be disposed of after being used once instead of being laundered or dry cleaned for re-use. Some examples of disposable undergarments include infant diapers, training pants, adult incontinence garments, feminine pants, etc.

Some pant-like disposable absorbent undergarments manufactured today resemble regular cloth underwear in that they have a waist opening and a pair of leg openings. Such pant-like disposable absorbent undergarments can be pulled up around the torso of a wearer in a similar fashion as regular cloth underwear. Still other pant-like disposable absorbent undergarments have an open or flat configuration and are designed to be placed adjacent to a wearer's torso and then rely upon one or more attachment tabs or fasteners to secure the undergarment around the wearer's torso. This design is beneficial for bed bound users who may be immobile or for children who need assistance in securing the undergarment in place. Still other adjustable, pant-like absorbent undergarments contain attachment means for opening and closing the waist opening after the undergarment has been positioned around the wearer's torso. This type of adjustable undergarment has an advantage in that the wearer does not have to remove outer clothing in order to check the status of the undergarment or to remove or apply the undergarment from their body.

One example of such an adjustable, pant-like disposable absorbent undergarment includes a pair of lines of weakness that a user must break to enable adjusting the fit of the undergarment. The lines of weakness usually extend from the waist opening to one of the leg openings and are designed to be broken either prior to positioning the undergarment around the user's torso or while the undergarment is already positioned around the wearer's torso. A pair of fastener assemblies is then utilized to refasten the undergarment so that it is snug about the wearer's torso.

It has been found that a major portion of each of the lines of weakness is visually hidden and some users cannot see them and thereby do not know that they are present. In addition, each line of weakness may be ergonomically hard to tear open by older adults, some of who may be suffering from arthritis. In addition, options that require tearing often indicate to users that the product is damaged or of poor quality when torn.

Typically, however, the types of consumer goods mentioned above are manufactured on a continuous basis on large scale manufacturing lines. Usually, various raw products or components are formed on, or integrated into, a continuous stream of material, which often includes a web of material that moves in a machine direction through and along the line. As such, it is important to maintain the integrity of the stream of material or web during the process so as to avoid costly downtime. In general, the web is pushed or pulled along the line, so as to put the web in tension. Accordingly, the formation of a fully severed panel, especially along a cross-direction, can increase the risk of breakage. Therefore, it is desirable to maintain the tensile strength of the stream of materials or web as it passes through the process.

An adjustable, pant-like disposable absorbent undergarment that includes a fully severed front panel is needed. Additionally, there is need to provide a method and apparatus for manufacturing pant-like disposable absorbent undergarment that includes fully severed front panel without causing a problem with the integrity of the stream of material or web during the process.

### SUMMARY

Generally, a method and apparatus for producing a pre-fastened adjustable pant-like disposable absorbent undergarment including a fully severed front body panel is disclosed. The method includes conveying a moving web for use as a body panel on a web stability control module. In addition, a fastener assembly is supplied that may be used to attach and connect portions of the moving web. Prior to attaching the fastener assembly to the moving web, a detection system is utilized to determine if there is a product defect on the fastener assembly. If no product defect is detected, the moving web is cut at a cutting station to completely separate a leading portion of the moving web and a trailing portion of the moving web at the cut. Then, the fastener assembly is attached to the moving web at a fastener assembly attachment station such that the leading portion and trailing portion remains bridged together by the fastener assembly. If a product defect is detected, then the moving web is not cut, and the discrete disposable absorbent undergarment is discarded.

In some embodiments, the detection system may include a fastener assembly detector, a moving web detector, or a combination thereof. In one embodiment, the detection system may indicate a product defect from a fastener assembly defect. In another embodiment, the detection system may further comprise indicating a product defect from a moving web defect. In some embodiments, the detection system automatically detects a product defect during machine start-up, machine shut-down, moving web splicing, and fastener assembly web splicing.

Web panel stability is an important aspect to maintaining the integrity of the process. The stability of the moving web can be maintained with use of the stability control module while conveying the moving web before the cutting station until after the fastener assembly is attached at the fastener assembly attachment station. The stability control module may include vacuum, surface roughness and/or needle/clamp engagement or other suitable means known to those skilled in the art to ensure stability of the web on the conveying means.

These features will be described in greater detail herein. Further, it is to be understood that both the foregoing general description and the following detailed description are exemplary and are intended to provide further explanation of the invention claimed.

### BRIEF DESCRIPTION

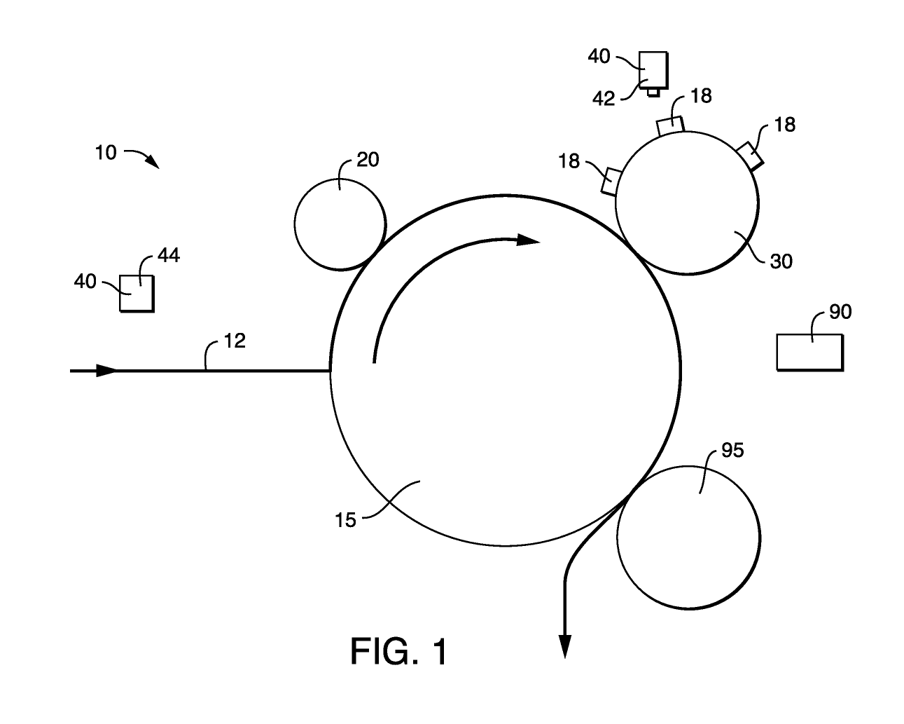

FIG. 1 depicts an apparatus for producing a pre-fastened adjustable pant-like disposable absorbent undergarment including a fully severed front body panel.

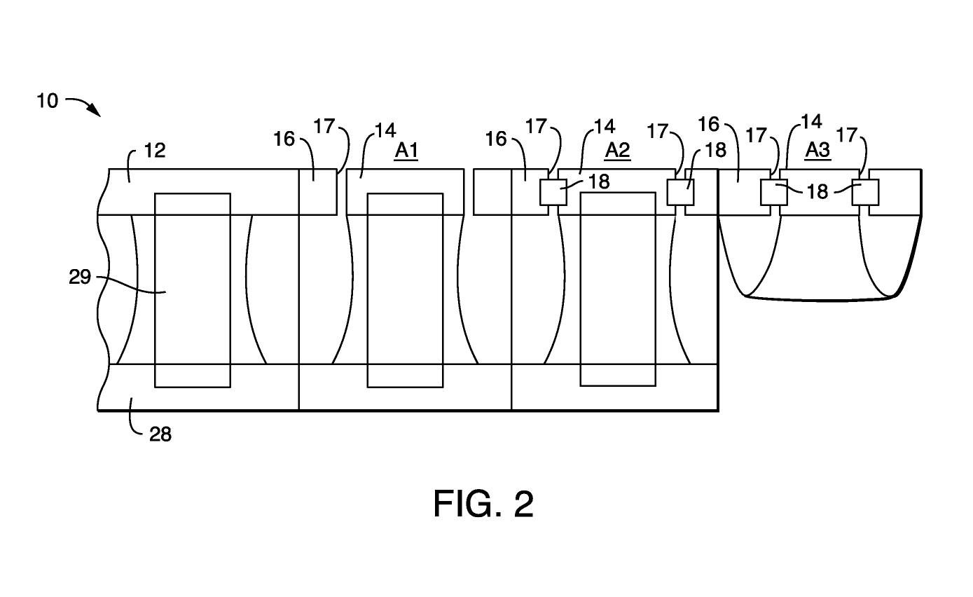

FIG. 2 depicts a method of producing a pre-fastened adjustable pant-like disposable absorbent undergarment including a fully severed front body panel.

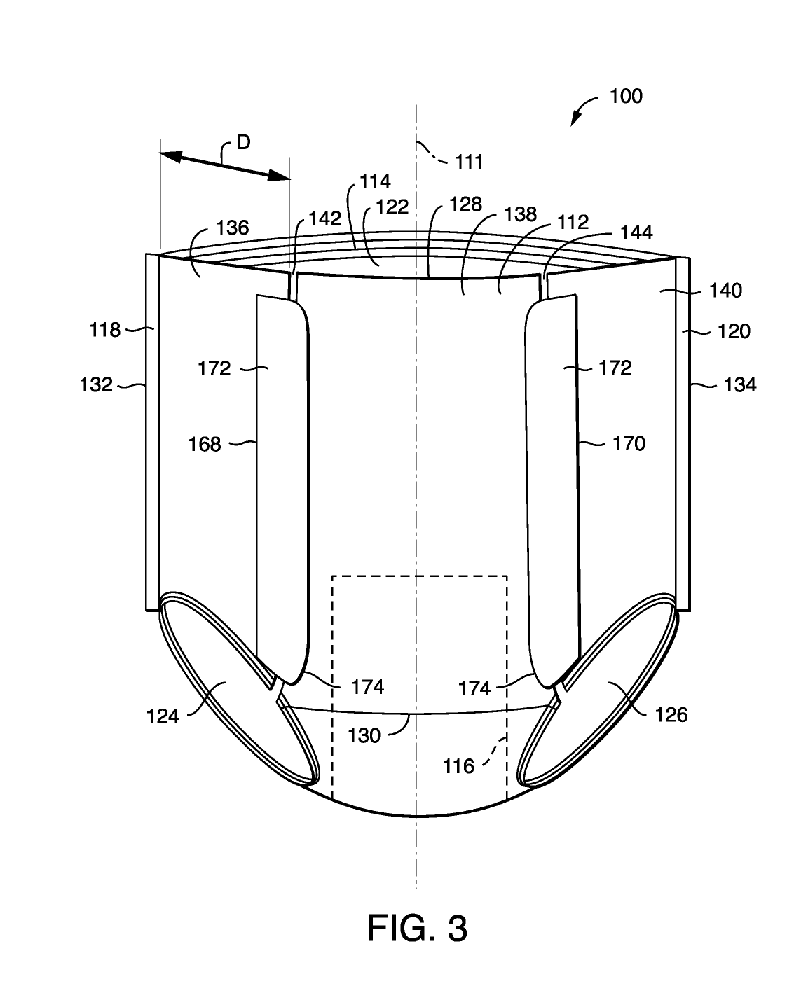

FIG. 3 depicts a front perspective view of one example of a garment, the garment shown in a pre-fastened, pant-like configuration.

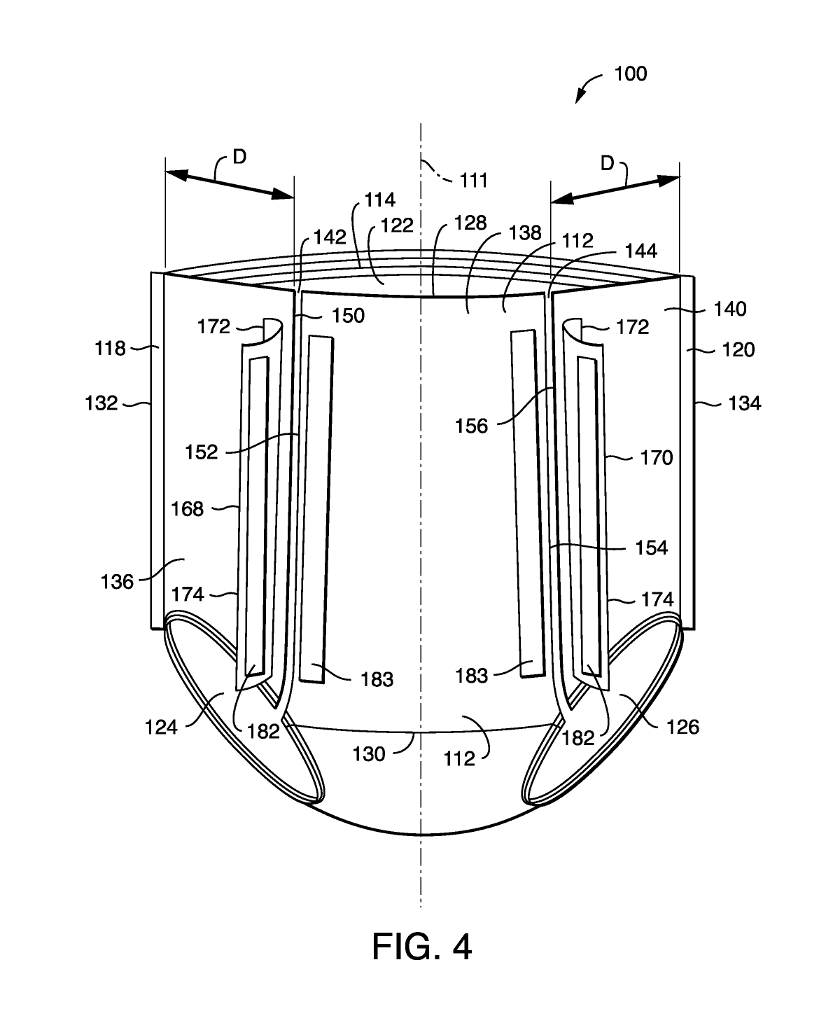

FIG. 4 depicts a front perspective view of one example of a garment, the garment shown in an unfastened, pant-like configuration.

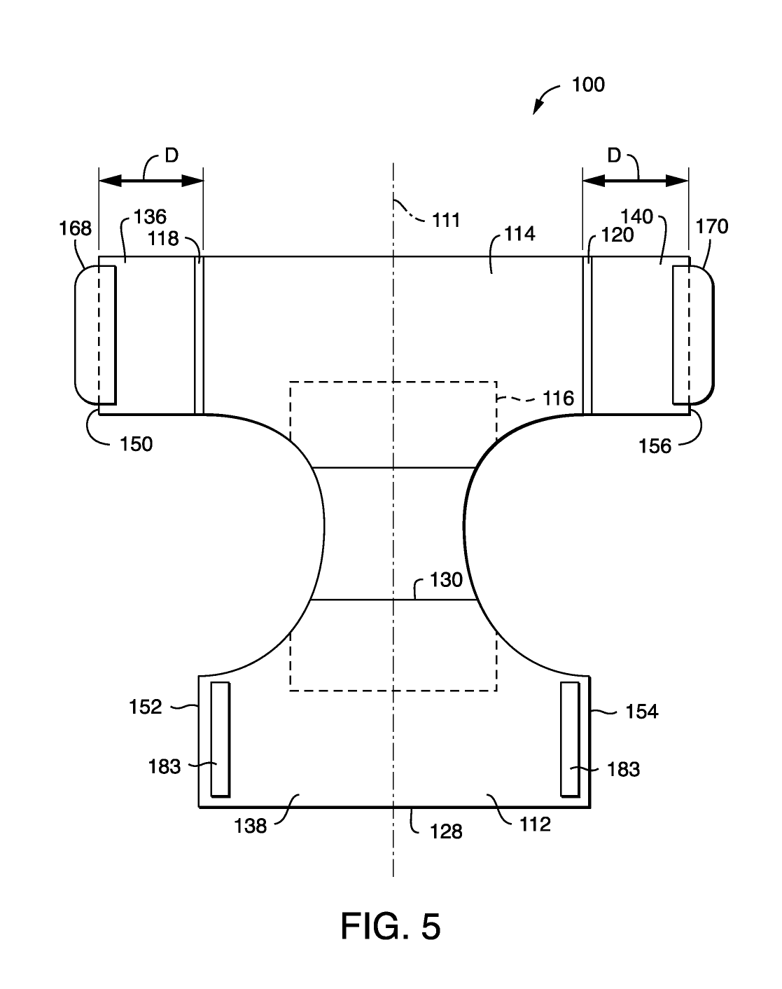

FIG. 5 depicts a plan view of the garment of FIG. 1, the garment shown in an unfastened, laid-open, relaxed configuration.

### DETAILED DESCRIPTION

Reference to the Figures shall be made in describing various embodiments. It should be noted that the embodiments depicted in the Figures and described herein are merely representative examples. The various embodiments are suitable for use in conjunction with disposable absorbent undergarments such as refastenable adult incontinence underwear, pre-fastened disposable diapers, refastenable disposable training pants or swim pants, refastenable disposable enuresis garments, and the like. For illustration purposes, various embodiments shall be described in conjunction with refastenable incontinence or enuresis underwear.

Within the context of this specification, each term or phrase below will include the following meaning or meanings.

The term “body side” should not be interpreted to mean in contact with the body of the user, but rather simply means the side that would face toward the body of the user when the garment is applied to the user, regardless of whether the absorbent garment is actually being worn by the user and regardless of whether there are or may be intervening layers between the component and the body of the user Likewise, the term “garment side” should not be interpreted to mean in contact with the garments of the user, but rather simply means the side that faces away from the body of the user when the garment is applied to the user, and therefore toward any outer garments that may be worn by the user, regardless of whether the absorbent garment is actually being worn by a user, regardless of whether any such outer garments are actually worn and regardless of whether there may be intervening layers between the component and any outer garment.

The term “machine direction” means the direction of flow as the various members and webs progress along the fabrication line and process. It should be understood that various separate members or webs can each be traveling in a machine direction, but with the various machine directions not necessarily being parallel or oriented in the same direction. For example, one web may be traveling along a first machine direction, which is substantially perpendicular to the travel of another web in a second machine direction.

The term “cross-direction” means the direction substantially perpendicular to the machine direction.

The term “downstream” means that one item is positioned more closely to the output or finished product end of the machine and/or process relative to another item. Conversely, the term “upstream” means that an item is positioned more closely to the input end of the machine or process relative to another item. For example, the output end is downstream of the input end, and vice versa, the input end is upstream of the output end.

The term “disposable absorbent undergarment” as used herein is an article that is intended to be worn by persons, including infants, toddlers or adults, which is designed for a single or temporary use and is meant to be disposed of after being used once instead of being laundered or dry cleaned for re-use.

The term “attached” refers to the joining, adhering, bonding, connecting, or the like, of two elements. Two elements will be considered to be attached together when they are attached directly to one another or indirectly to one another, such as when each is directly attached to intermediate elements.

The term “disposable” refers to articles which are designed to be discarded after a limited use rather than being laundered or otherwise restored for reuse.

The term “elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

The terms “longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

Generally, a method and apparatus for producing a pre-fastened adjustable pant-like disposable absorbent undergarment including a fully severed front body panel is disclosed.

The method includes conveying a moving web for use as a body panel on a web stability control module. In addition, a fastener assembly is supplied that may be used to attach portions of the body panel. A detection system is utilized to determine if there is a product defect on the fastener assembly. If no product defect is detected, the moving web is cut at a cutting station to completely separate a leading portion of the moving web and a trailing portion of the moving web at the cut. Then, the fastener assembly is attached to the moving web at a fastener assembly attachment station such that the leading portion and trailing portion remains bridged together by the fastener assembly. If a product defect is detected, then the moving web is not cut, and the disposable absorbent undergarment is discarded.

Referring to FIGS. 1 and 2, a method and apparatus 10 for producing a pre-fastened adjustable pant-like disposable absorbent undergarment is illustrated. A moving web 12 is shown as moving in a machine direction along a process line. The moving web 12 may be used as a front body panel, a back body panel, or both in the disposable absorbent undergarment.

The moving web 12 can be formed from a single piece of material or can be formed as a laminate consisting of two or more layers. The layers of the laminate can be of the same material or different material. In one embodiment, a laminate is formed from a first layer and a second layer. Sandwiched between the first and second layers are two or more elastic strands. Desirably, from two to about a hundred elastic strands can be utilized in the moving web 12 depending upon the overall size of each panel. The elastic strands can be formed from LYCRA, or a similar material. LYCRA is a trademark of INVISTA (Wichita, Kans.). The diameter and/or cross-sectional configuration of the elastic strands, the decitex (weight in grams per 10,000 meters) of the elastic strands, and the tension imparted into the elastic strands can all be varied to suit one's particular product needs. The exact number of elastic strands that are utilized should be sufficient to ensure that the disposable absorbent undergarment snuggly conforms to the wearer's torso.

The elastic strands can be coated with an adhesive. By adhesively coating each of the elastic strands, instead of coating a major portion of the inner surface of at least one of the first and second layers, softer moving webs 12 respectively, can be obtained. Wearers of disposable absorbent undergarments prefer a product that has a softer feel since it is more like underwear.

In other embodiments, the elastomeric nonwoven material comprises an elastomeric film sandwiched between two nonwoven facing layers. U.S. Pat. No. 7,803,244 to Siqueira et al., hereby incorporated by reference, discloses particular examples of elastomeric nonwoven composites suitable for use in the adjustable undergarment described herein.

It should be noted that the moving web 12 can be formed from a breathable or a non-breathable material. Desirably, the moving web 12 is formed from a breathable material or a material that is treated or processed to be breathable. Spunbond and bonded carded webs are two breathable materials that work well as moving web 12 in disposable absorbent undergarments. Bonded carded webs are produced and commercially sold by a variety of vendors. Other materials that can be used to form the moving web 12 include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

In addition, a fastener assembly 18 is provided to be attached to the moving web 12. The fastener assembly 18 may be attached using a fastener attachment station 30. For example, in one embodiment, the fastener assemblies 18 can be rotated using a cam action rotator. The rotator includes a plurality of transfer segments, which can have a vacuum applied thereto, that engage the fastener assemblies. Coupler arms connect the transfer segments and a drive ring. The coupler arm includes a cam end having a cam follower that follows the profile of a cam mechanism. The profile of the cam mechanism can be readily changed to change the desired speed output and pitch of the fastener assemblies. If the successive fastener assemblies are separated by a perforation, the transfer segment breaks the perforation as it engages the fastener assembly 18 and moves away from the next fastener assembly, which is engaged by a next transfer segment. Alternatively, the fastener assemblies are already cut all of the way through, and the rotator merely moves, or separates, and rotates the fastener assemblies. In yet another alternative embodiment, the fastener assemblies are cut and separated by the transfer segments. In other embodiments, the fastener assembly station may be a slip cut module. Various alternative methods and apparatus for applying fastener members to an absorbent article are disclosed in U.S. Pat. No. 6,730,188 entitled “Method and Apparatus For Assembling Refastenable Absorbent Garments,” U.S. Pat. No. 6,743,321 entitled “Method and Apparatus For Assembling Refastenable Absorbent Garments,” U.S. Pat. No. 6,686,626 entitled “Method and Apparatus For Assembling Refastenable Absorbent Garments,” U.S. Pat. No. 6,682,626 entitled “Method and Apparatus For Assembling Refastenable Absorbent Garments,” U.S. Pat. No. 6,712,922 entitled “Multiple Component Web,” U.S. Pat. No. 6,783,487, entitled “Pant-Type Personal Care Articles, and Methods of Making and Using Such Personal Care Articles,” U.S. Pat. No. 6,454,888 entitled “Methods of Changing Size of Pant-Type Personal Care Articles Outputted from a Manufacturing Process,” the entire disclosures of which are hereby incorporated by reference.

Prior to attaching the fastener assembly 18 to the moving web 12, a detection system 40 is utilized. The detection system 40 may be a visual detection system such as a camera or photo-eye. The detection system 40 may inspect the moving web 12 or the fastener assembly 18 to ensure there is not a product defect. In one embodiment, the detection system 40 includes a fastener assembly detector 42. The fastener assembly detector 42 may visually inspect the fastener assembly 18 to ensure a proper functioning fastener assembly 18. In another embodiment, the detection system 40 may further include a moving web detector 44. The moving web detector 44 may visually inspect the moving web 12 to ensure a proper functioning moving web. If no product defect is detected, the moving web 12 is passed through a cutting station 20 at a first position A1.

Product defects may include a variety of different things that may result in the discrete pre-fastened adjustable pant-like disposable absorbent undergarment being defective. In addition, a product defect is a product or process condition that would lead to a web break in the moving web because the fastener assembly is not placed properly. For example, the product defect may be a defective fastener assembly. A defective fastener assembly may be a missing component on the fastener assembly, a misaligned fastener assembly, a problem with the adhesive on the fastener assembly, a cut or tear in the fastener assembly, or other fastener assembly issues known to those skilled in the art. In addition, the product defect may be a defective moving web. A defective moving web may be a missing component on the moving web such as an insert, leg elastics, flaps, waistband, a misaligned moving web, a cut or tear in the moving web, or other moving web issues known to those skilled in the art.

Product defects may also occur when making a splice in a fastener assembly web or the moving web. For example, splicing could result in a bad fastener assembly or moving web, too much thickness, or missing component on the fastener assembly or moving web resulting in a defective product. Additionally, product defects may occur during machine start-up or shut-down. For example, machine start-up or shut-down fastener assemblies may be lost, adhesive issues such as cold or hot adhesive, friction changes on speed changes can all result in fastener assembly damage. Therefore, products made during machine shut-down, machine start-up, moving web splicing and fastener assembly splicing may be considered to automatically have product defects by the detection system 40.

If a product defect is detected, then the moving web 12 is not cut, and the pant-like disposable absorbent undergarment is discarded. Not cutting the moving web 12 can be done in a number of different ways. In one embodiment, the cutting blade may be disengaged. In another embodiment, the relationship between the knife and anvil may be phased to prevent cutting of the knife. This may be done by phasing the cutting knife off the anvil, phase the anvil, or drum away from the knife, or reducing or eliminating pressure between knife and anvil. In an alternative embodiment, if the cutting mechanism is a laser, the laser may be shut off. Any other suitable means known to those skilled in the art may be used to prevent the moving web 12 from being cut.

Alternatively, if no product defect is detected, the moving web 12 is passed through a cutting station 20 at a first position. The cutting station 20 forms a cut 17 in the moving web 12 to define a leading portion 14 of the web and a trailing portion 16 of the web connected at the cut 17, as shown for example in FIG. 2. In a desirable embodiment, the cutting station 20 forms a cross-directional line for the cut 17, which is preferably linear, in the web. Desirably, the cut 17 can extend across the entire cross-direction width of the web. In some embodiments, the cut 17 can be tapered relative to the longitudinal axis, if desired. In addition, the cut 17 can also be curved.

In one desirable embodiment, the cutting station 20 is configured as a perforator having a knife roll and an anvil roll. Alternatively, the cutting station 20 can be configured with a laser, water jet, or other types of cutters known to those skilled in the art. In other alternative embodiments, the cutting station 20 can comprise a device for applying heat, thermal energy or ultrasonic energy to the web so as to cut it at specific locations. In other preferred embodiments, the cutting station 20 can include a chemical applicator that applies various chemicals, including, for example, water, to the web to cut it at specific locations. In yet another alternative embodiment, the apparatus applies a speed differential to the web so as to cut the web. Of course, it should be understood that the cutting station 20 can also be configured from combinations of one or more of the above-referenced devices.

After cutting the web, the moving web 12 is passed through the fastener attachment station 30 at a second position A2. The fastener attachment station 30 attaches a fastener assembly 18 over the cut 17 that defines a leading portion 14 of the moving web 12 and a trailing portion 16 of the moving web 12 together with the fastener assembly 18. The fastener assembly 18 will help maintain the integrity of the moving web 12 as it continues through the remainder of the process.

Web panel stability is an important aspect to maintaining the integrity of the process. The stability of the moving web 12 can be maintained with use of a stability control module 15 while conveying the moving web 12 that engages the moving web 12 before the cutting station 20 until after the fastener assembly 18 is attached at the fastener assembly attachment station 30. Desirably, the stability control module 15 may be a vacuum drum. Alternatively, the stability control module 15 may include vacuum, surface roughness and/or needle/clamp engagement or other suitable means known to those skilled in the art to ensure stability of the web on the conveying surface.

Desirably, the cutting station 20, stability control module 15, and the fastener assembly attachment station 30 are an integral step in a manufacturing process. For example, the stability control module 15 may be a vacuum drum. While the moving web 12 is conveyed on the vacuum drum, it passes through the cutting station 20 to fully separate the web. The vacuum drum maintains the stability of the moving web 12 after it is cut. Soon after cutting, the moving web 12 is conveyed on the vacuum drum through the fastener assembly attachment station 30. All of these components are incorporated into a single step in the manufacturing process. In other embodiments, these steps may be included as separate components on a manufacturing line.

Prior to removing the moving web 12 from the web stability control module 15, a backup detection system 90 may be utilized. The backup detection system 90 may be a visual detection system such as a camera or photo-eye. The backup detection system 90 may inspect the moving web 12 to ensure proper placement of the fastener assembly 18. If a cut was made on the moving web 12 and the fastener assembly 18 is not present or misaligned, then a fastener replacement station 95 may be engaged. In some embodiments, if the fastener assembly 18 is misaligned, the product is simply is discarded and the fastener replacement station 95 is not engaged.

If engaged when the backup detection system 90 detects an improperly applied fastener assembly 18, the fastener replacement station 95 applies a repair patch for the fastener. The repair patch maintains integrity of the moving web 12 on the process and prevents the process downtime. In one embodiment, the fastener replacement station 95 applies a replacement fastening material such as adhesive tape or a Velcro hook material. In another embodiment, the fastener replacement station 95 will shift the leading portion 14 of the moving web 12 to overlap with the trailing portion 16 of the moving web 12 and bond the leading portion 14 to the trailing portion 16. In another embodiment, the fastener replacement station 95 will bond a material over the cut. Once a repair patch is applied over the cut, the discrete pant-like absorbent article is culled and discarded.

If the backup detection system 90 detects proper placement of a fastener assembly 18, the manufacturing process continues and the discrete pre-fastened adjustable pant-like absorbent article is completed. In one embodiment, illustrated in FIG. 2 as referenced by FIG. 3, the moving web 12 is used as a front body panel 112 and bonded to a rear body panel web 28 at side seams 118, 120, wherein the rear body panel web 28 is positioned over the moving web 12 by folding a crotch portion 29 joining or bridging between the moving web 12 and rear panel web 28 at a third location A3.

Referring to FIGS. 3-5, an adjustable pant-like disposable absorbent undergarment 100 having a longitudinal axis 111 made using the method described herein is shown. The adjustable pant-like disposable absorbent undergarment 100 is designed to absorb liquid, semi-solid and/or solid waste discharged from a human being. The adjustable pant-like disposable absorbent undergarment 100 is designed to absorb and/or retain one or more bodily discharges of waste material such as urine, perspiration, excrement, feces, menses, menstrual fluid, as well as other liquid and/or solid waste.

The adjustable pant-like disposable absorbent undergarment 100 includes a front body panel 112, a back body panel 114 and an absorbent assembly 116 secured to the front and back body panels, 112 and 114 respectively. The front and back body panels, 112 and 114 respectively, are joined together by a pair of seams 118 and 120 to form a waist opening 122 and a pair of leg openings 124 and 126.

The front body panel 112 includes a waist edge 128, a crotch edge 130 and a pair of side edges 132 and 134. In a three piece construction wherein an absorbent assembly 116 is secured between the front body panel 112 and the back body panel 114, the crotch edge 130 is well defined. In absorbent undergarments of a different construction, the crotch edge 130 can be an imaginary line transversely drawn between the pair of leg openings 124 and 126 at a location where one considers the front body panel 112 to end. The exact size and configuration of the front body panel 112 can vary to suit a wearer's particular needs. The front body panel 112 has a first side section 136, a middle region 138 and a second side section 140. The front body panel 112 is completely severed between both the first side section 136 and the middle section 138 and the second side section 140 and the middle section 138 creating a first fully severed region, or gap, 142 and a second fully severed region 144. The first side section 136 is aligned adjacent to the side edge 132 at its leading edge forming the seam 118 and the second side section 140 is aligned adjacent to the side edge 134 at its leading edge forming the seam 118. The middle section 138 has a first edge 152 and a second edge 154 and is located between the first and second side sections, 136 and 140 respectively. The terminal edge 150 of the first side section 136 is aligned adjacent to the first edge 152 of the middle section 138 and the terminal edge 150 of the second side section 140 is aligned adjacent to the second edge 154 of the middle section 138. As depicted, the middle section 138 is centrally located and is bifurcated by the longitudinal axis 111 at a midpoint of the front body panel 112.

The first and second fully severed regions, 142 and 144, can be linear or non-linear in configuration. In FIGS. 4-5, the first and second fully severed region, 142 and 144, are shown having a linear or straight configuration. The first and second fully severed regions, 142 and 144, extend longitudinally from approximately the waist edge 128 down to approximately one of the leg openings 124 or 126. The first and second fully severed regions, 142 and 144, can be aligned parallel to the longitudinal axis 111 or be angled thereto. For example, the first and second fully severed regions, 142 and 144, can be tapered relative to the longitudinal axis 111, if desired. In addition, the fully severed regions, 142 and 144, can also be curved.

Still referring to FIGS. 4-5, each of the first and second fully severed regions, 142 and 144, extend from approximately the waist edge 128 of the front body panel 112 to one of the pair of leg openings 124 and 126. Another way of describing this is to say that the first and second fully severed regions, 142 and 144, extend from approximately the waist edge 128 of the front body panel 112 to approximately the crotch edge 130 of the front body panel 112.

Many adjustable, pant-like disposable absorbent undergarments include a pair of lines of weakness to break to allow for the individual to adjust the size of the pants. This type of adjustable disposable undergarment including the lines of weakness requires an amount of force needed to break the lines of weakness. The pre-fastened adjustable, pant-like disposable absorbent undergarments 100 described herein have the first and second fully severed regions, 142 and 144, that require no force to break allowing for easier use by an elderly individual, caretaker or parent caring for a child.

Still referring to FIGS. 4-5, each of the first and second fully severed regions, 142 and 144, is shown being aligned parallel to one of the pair of seams 118 and 120. Such an arrangement provides for an aesthetically pleasing appearance to the front body panel 112.

Desirably, in the pre-fastened condition, the first side section 136 and the middle section 138 of front body panel 112 do not overlap, and the second side section 140 and the middle section 138 of the front body panel 112 do not overlap in the severed regions 142, 144. In one exemplary embodiment, a first gap may be formed in the first severed region 142 between the first side section 136 and the middle section 138 of the front body panel 112 in the pre-fastened condition. In this embodiment, a second gap may be formed in the first severed region 142 between the second side section 140 and the middle section 138 of the front body panel 112 in the pre-fastened condition. In another exemplary embodiment, a terminal or outer edge 150 of the first side section 136 abuts against the first edge 152 of the middle section 138 of the front body panel 112 in the pre-fastened condition. In this embodiment, a terminal or outer edge 150 of the second side section 140 abuts against the second edge 154 of the middle section 138 of the front body panel 112 in the pre-fastened condition. In another embodiment, a gap may be formed between one side section and the middle section and the other side section and middle section abut against each other. If a gap is formed between the side sections 136 and 140, and the middle section 138, the gap will desirably be less than 10 mm.

Referring again to FIGS. 4-5, the front body panel 112 further includes a pair of fastener assemblies 168 and 170. Each fastener assembly 168 and 170 includes a first portion 172 and a second portion 174. The first portion 172 of the fastener assembly 168 can be permanently secured to the first side section 136 of the front body panel 112 and the first portion 172 of the other fastener assembly 170 can be permanently secured to the second side section 140 of the front body panel 112.

In the embodiment shown in FIGS. 4-5, the first portions 172 of the fastener assemblies 168 and 170 can be securely attached using an adhesive, heat, pressure, a combination of heat and pressure, an ultrasonic bond, a chemical bond or by other means known to those skilled in the art.

Each of the second portions 174 of the fastener assemblies 168 and 170 are depicted as extending almost the entire length of the fully severed portions 142 and 144. This means at least 50% of the distance between the waist edge 128 and the leg opening 124 are covered by the fastener assembly 168. Desirably, the first fastener assembly 168 and second fastener assembly 170 extend at least 65% of the distance between the waist edge 30 and the leg opening 124. More desirably, the first fastener assembly 168 and second fastener assembly 170 extend at least 75% of the distance between the waist edge 128 and the leg opening 124. Even more desirably, the first fastener assembly 168 and second fastener assembly 170 extend at least 85% of the distance between the waist edge 128 and the leg opening 124. Even more desirably, the first fastener assembly 168 and second fastener assembly 170 completely extend at least 95% of the distance between the waist edge 128 and the leg opening 124. The second portions 174 of the first fastener assembly 168 can bridge across the first fully severed region 142 and the second portion 174 of the other fastener assembly 170 can bridge across the second fully severed region 144. By extending substantially the entire length of and bridging the fully severed portions, 142 and 144, the fastener assemblies 168, 170 cover the fully severed portions and provide a more underwear like appearance. The second portions 174 of the fastener assemblies 168 and 170 can be releasably attached to the middle section 138 of the front body panel 112.

Alternatively, it should be evident to those skilled in the art that the first portion 172 of each of the fastener assemblies 168 and 170 could be permanently attached to the middle section 138. In this embodiment, the second portions 174 of the fastener assemblies 168 and 170 can be releasably attached to the side sections 136, 140 of the front body panel 112. In an alternative embodiment, both the first portion 172 and the second portion 174 may be releasably attached. The fastener assembly 168 may be constructed of a non-extensible or an elastomeric material.

Referring now to FIGS. 4-5, each of the second portions 174 of the fastener assemblies 168 and 170 has an inner surface 180 that contains a fastener 182. The fastener 182 can be a mechanical fastener 182. In FIGS. 9 and 10, the mechanical fastener 182 is shown as a plurality of fine hooks, such as VELCRO hooks. VELCRO is a trademark of Velcro USA, Inc. (Manchester, N.H.). The hooks are designed to easily engage and be removed from a material wherein the material has a loose weave pattern or the fibers forming the material will allow the hooks to be attached to them. The mating material is commonly referred to as the loop member of a hook and loop fastener. The middle section 138 of the front body panel 112 may be formed of such a material. When the hooks engage into the middle section 138, a secure but releasable fastener is formed. The hooks can be easily removed from the loop material by pulling the edge of the fastener outward away from the middle section 138. Alternatively, as illustrated in FIGS. 9 and 10, a fastening component 183 such as a loop material or loose fibers that may be placed anywhere onto the middle section 138 to facilitate the fastening component 183. Therefore, hook and loop fasteners are referred to as being releasable and can be fastened and released several times. In alternative embodiments, the mechanical fastener 182 constructed of hooks may be placed on to the middle section 138 of the front body panel 112. In this embodiment, the fastening component 183 constructed of a loop material or loose fibers would be placed on the fastener assemblies 168, 170. Both the mechanical fastener 182 and the fastening component 183 may be integral or separately attached.

Referring again to FIGS. 3-5, one can see that FIG. 3 depicts the pair of fastener assemblies 168 and 170 being securely fastened to the middle section 138 of the front body panel 112. If the wearer of the disposable absorbent undergarment 100 or a caregiver wishes to inspect the undergarment 100, he or she would open the pair of fastener assemblies 168 and 170 to the position shown in FIG. 4. Since the first and fully severed portions, 142 and 144 are already broken, the middle section 138 of the disposable absorbent undergarment 100 can be easily moved outward away from the wearer's torso. The wearer can then inspect the absorbent assembly 116 to see if it needs to be changed. If so, the disposable absorbent undergarment 100 can be removed from about the wearer's torso and be replaced by another undergarment. If the absorbent assembly 116 is still capable of accepting additional body fluid, the middle section 138 is moved back against the wearer's torso and the pair of fastener assemblies 168 and 170 is refastened to the middle section 138.

The horizontal distance to each of the first and second fully severed regions, 142 and 144, and corresponding first and second fastener assemblies, 168 and 170, as located relative to the pair of side seams 118 and 120 can also vary. A distance “D” is depicted in FIGS. 4-5 which represents the distance between each of the pair of seams 118 and 120 and the corresponding first and second fully severed regions, 142 and 144 respectively. One can increase the distance “D” by moving the first and second fully severed regions, 142 and 144 respectively, away from the pair of seams 118 and 120. When one increases the distance “D”, one may find that it is easier for the wearer of the disposable absorbent undergarment 100 to visually see and identify the first and second fully severed regions, 142 and 144, when the user looks down at the front body panel 112. In some embodiments, the first fastener assembly 168 and the second fastener assembly 170 are located near the midpoint between the side seams, 118 and 120, and the longitudinal axis 111 of the front panel 112. Desirably, the first fastener assembly 168 and second fastener assembly 170 are located between 30% and 70% of the distance between the side seam, 118 and 120, and the cross-sectional midpoint 111 of the front panel 112. More desirably, the first fastener assembly 168 and second fastener assembly 170 are located between 35% and 45% of the distance between the side seams, 118 and 120, and the cross-sectional midpoint 111 of the front body panel 112. A manufacturer is free to vary the distance “D” to best suit the size and shape of a particular disposable absorbent undergarment 100 to make sure that the fastener is on the front of the product rather than the side of the product. If the fully severed regions 142, 144 are not parallel to the side seams 118, 120, the midpoint of the fully severed regions 142, 144 in the longitudinal direction may be used to measure “D”.

Placement near the midpoint between the side seam and the longitudinal axis 111 of the front body panel 112 facilitates enhanced donning and removal when experiencing a reduced range of motion as well as locating the fastener in a location which is easier for the consumer to see.

The placement of the fastener assemblies 168 and 170 in the front of the product optimizes the ability of the user to fasten the product, while minimizing potential pop-opens during use due to movement of the legs of the user. The placement of the fasteners can be measured in both an unstretched (out of the bag) state or stretched (as in use).

It should be understood that various other embodiments, modifications, and equivalents to the embodiments of the absorbent article described herein which, after reading the description herein, may suggest themselves to those skilled in the art without departing from the scope and spirit of the present claims.

What is claimed is:

1. A method of producing discrete pre-fastened adjustable disposable absorbent garments, comprising:
a. conveying a moving web for use as a body panel on a web stability control module;
b. providing a fastener assembly to be attached to the moving web; and
c. inspecting for a product defect on the fastener assembly with a detection system,
i. wherein if a product defect is not present, cutting the moving web to completely separate a leading portion of the moving web and a trailing portion of the moving web at the cut; and attaching a fastener assembly to the moving web such that the leading portion and trailing portion remains bridged together by the fastener assembly;
ii. wherein if a product defect is present, the moving web is not cut and the discrete disposable absorbent garment is discarded.

2. The method of claim 1 wherein the detection system comprises a fastener assembly detector to detect a fastener assembly defect.

3. The method of claim 1 further comprising inspecting for the product defect on the moving web with a moving web detector to detect a moving web defect.

4. The method of claim 1 wherein the product defect is selected from a fastener assembly defect, a moving web defect or a combination thereof.

5. The method of claim 1 wherein the detection system detects a product defect automatically during machine start-up, machine shut-down, moving web splicing, and fastener assembly web splicing.

6. The method of claim 1 wherein the web stability control module is selected from vacuum engagement, surface roughness, clamp engagement, needle engagement and combinations thereof.

7. The method of claim 1 wherein the web stability control module, a web cutting station, and the fastener assembly attachment station comprise an integral component in a manufacturing process.

8. The method of claim 5 wherein the web stability control module is a vacuum drum.

9. The method of claim 1 further comprising inspecting the cut web with a backup detection system to ensure proper placement of a fastener assembly, and wherein incorrect placement occurs, attaching a replacement fastening material over the cut in the moving web.

10. An apparatus for producing a pre-fastened adjustable pant-like disposable absorbent undergarment comprising:
a. a web stability control module for conveying a moving web for use as a body panel;
b. a product defect sensor for detecting a product defect in the disposable absorbent undergarment;
c. a web cutting station for completely separating the moving web at a leading portion of the moving web and a trailing portion of the moving web; and
d. a fastener attachment station for bridging the connected the leading portion of the moving web and the trailing portion of the moving web together with a fastener assembly.

11. The apparatus of claim 10 wherein the detection system comprises a fastener assembly detector to detect a fastener assembly defect.

12. The apparatus of claim 10 wherein the detection system further comprises a moving web detector to detect a moving web defect.

13. The apparatus of claim 10 wherein the detection system detects a product defect automatically during machine start-up, machine shut-down, moving web splicing, and fastener assembly web splicing.

14. The apparatus of claim 10 wherein the web stability control module is selected from vacuum engagement, surface roughness, clamp engagement, needle engagement and combinations thereof.

15. The apparatus of claim 10 wherein the web stability control module is a vacuum drum.

16. The apparatus of claim 10 wherein the web stability control module, a web cutting station, and the fastener assembly attachment station comprise an integral component in a manufacturing process

17. The apparatus of claim 10 further comprising a backup detection system to ensure proper placement of a fastener assembly, and wherein incorrect placement occurs, further comprising a replacement fastening material station to over the cut in the moving web.

18. A method of producing discrete pre-fastened adjustable disposable absorbent garments, comprising:
a. conveying a moving web for use as a body panel on a web stability control module;
b. providing a fastener assembly to be attached to the moving web;
c. inspecting for a product defect on the fastener assembly with a detection system; and
d. wherein if a product defect is not present, cutting the moving web to completely separate a leading portion of the moving web and a trailing portion of the moving web at the cut; and attaching a fastener assembly to the moving web such that the leading portion and trailing portion remains bridged together by the fastener assembly.

19. The method of claim 18 wherein the detection system comprises a fastener assembly detector to detect a fastener assembly defect.

20. The method of claim 18 further comprising inspecting for the product defect on the moving web with a moving web detector to detect a moving web defect.

21. The method of claim 18 wherein the detection system detects a product defect automatically during machine start-up, machine shut-down, moving web splicing, and fastener assembly web splicing.

22. A method of producing discrete pre-fastened adjustable disposable absorbent garments, comprising:
a. conveying a moving web for use as a body panel on a web stability control module;
b. providing a fastener assembly to be attached to the moving web;
c. inspecting for a product defect on the fastener assembly with a detection system; and
d. wherein if a product defect is present, the moving web is not cut and the discrete disposable absorbent garment is discarded.

23. The method of claim 22 wherein the detection system comprises a fastener assembly detector to detect a fastener assembly defect.

24. The method of claim 22 further comprising inspecting for the product defect on the moving web with a moving web detector to detect a moving web defect.

25. The method of claim 22 wherein the detection system detects a product defect automatically during machine start-up, machine shut-down, moving web splicing, and fastener assembly web splicing.
